(19) United States
(12) Patent Application Publication
(10) Pub. No.: US 2003/0029474 A1
(43) Pub. Date: Feb. 13, 2003

(54) Method and appratus for cleaning dental instruments such as endodontic files or the like

(76) Inventors: David Gibbs, Ottawa (CA); Neil Teitelbaum, Ottawa (CA)

Correspondence Address: Neil Teitelbaum, 834 Colonel By Drive, Ottawa, ON, K1S 5C4, CA

(21) Appl. No.: 09/927,237

(22) Filed: Aug. 13, 2001

(51) Int. Cl. B08B 3/12; B08B 6/00; B08B 7/00; A61L 2/00; A61C 5/02

(52) U.S. Cl. 134/1; 134/6; 134/34; 134/184; 422/20; 433/224

(57) ABSTRACT

A portable dental system and method is disclosed for cleaning endodontic files and the like during a operation such as a root canal procedure. This allows a dentist to clean files in situ while performing the removal of root material. As the file is withdrawn from the patients mouth by the dentist, it can be inserted into a sealed container through the lid and can be cleaned ultrasonically and immediately withdrawn and reinserted into a patients tooth canal for further removal of tooth and root material. Debridement of the files ensures less likelihood the file will break within the canal; furthermore, since no passing off to or cleaning the tip by an assistant is required, there is less likelihood of inadvertent skin puncturing and infection of the dentist or assistant. The sealed disposable container housing a cleaning fluid is insertably removable from the base, which supports it and provides a source of ultrasonic energy thereto.

### FIELD OF THE INVENTION

This invention is useful in the dental and medical arts. The invention relates to a container configured to disinfect a portion of a dental or medical instrument in at least an aseptic condition during a dental procedure.

### BACKGROUND

In the past when engaging in dental procedures, a prime concern was that the dental instruments employed do not introduce infection into the mouth of the patient. Avoiding the introduction of microorganisms to the area of a tooth being treated is particularly important when performing root canal therapy since infections cause significant problems in root canals. In fact, the inability to successfully counteract harmful microorganisms in a root canal is the primary reason that infected or potentially infected tissue is removed from a root canal during endodontic or root canal therapy. Accordingly, it is important to avoid introducing any additional microorganisms into the root canal.

As part of the root canal therapy, the practitioner typically employs endodontic files to file the root canal and remove infected material. Endodontic files typically comprise a thin, distal insertion end and a proximal gripping end held by a practitioner or inserted into mechanical instruments such as a drill. The thin distal insertion end is often a delicate, flexible tip with sharp edges to enable efficient cleaning of the root canal.

To reduce the possibility of causing infection while working within a dental patient's mouth, it is vital that endodontic files and other dental instruments be maintained in a disinfected environment before and during use. In light of this need for a sterile technique, a variety of different disinfecting dental instrument containers have been developed.

For example, certain dental instrument containers have been produced which are capable of being placed in an autoclave, thereby sterilizing the instruments in the container. Other dental instrument containers have been developed which contain a disinfecting solution for placement of dental instruments into the solution. U.S. Pat. No. 3,248,167 to Friedman, for example, discloses a magnetic dental burr holder. The magnetic burr holder features a box-like container which contains a sterilizing solution. The container includes a hinged lid having a magnetized inner surface. Dental burrs are demountably coupled to the magnetized inner surface such that when the lid is closed, the tips of the dental burrs are immersed in the sterilizing solution.

However, magnetized containers may only be employed to maintain certain metal instruments within a solution. The magnetic container could not be employed to maintain a plastic instrument or the plastic portion of an instrument in a desired position. Additionally, use of metal containers can also be disadvantageous due to their relative weight and due to their cost, which may prevent their use on a disposable basis.

Other containers include a well or reservoir for containing both a sterilizing solution and an instrument immersed in the solution, such as the container disclosed in U.S. Pat. No. 4,306,862. However, a mere reservoir fails to maintain a portion of the dental instrument outside of the reservoir. Thus, when a practitioner desires to grasp the dental instrument, the practitioner must immerse the practitioner's fingers or another dental instrument into the sanitary solution, potentially permitting infectious material to pollute the solution.

U.S. Par. No. 4,232,784 to Hesselgren discloses a stand for instruments for medical use. This stand features sheets of paper arranged vertically close together. The sheets are held close together by the stand. Dental instruments are inserted between the sheets for storage during a sterilization process. To protect the dental instruments from corrosion during a sterilization process, the sheets are impregnated with a corrosion inhibiter. The sterilization process involves either autoclave sterilization in saturated water vapor at 120.degree. C. to 130.degree. C. or by means of dry sterilization at 180.degree. C.

One of the problems associated with a paper stand as disclosed in U.S. Pat. No. 4,232,784 is that the instrument must be placed between the paper sheets. The practitioner must therefore take time to ensure that the instrument is not directed into the paper which may potentially bend the instrument. The corrosion inhibitor may cause the papers to clump together, thereby losing their ability to receive a dental instrument. Furthermore, if papers on one side clump together, the papers on an opposing side could be separated too broadly, such that the dental instruments would slip between the cracks, precluding convenient gripping of the gripping end. The paper could also flake off onto the tips when wet or when ripped from the container. Additionally, as stated at column 3, lines 3-6 after having been used a number of times the instrument stand is considered expended. The stand cannot be used to maintain an instrument in a desired position after a few insertions of instruments as the instruments either break or weaken the fiber matrix of the paper or due to the debilitating impact of either the corrosion inhibitor or sterilization process on the paper.

As an additional difficulty within the art, typical dental instrument containers are complex and expensive structures containing a variety of reservoirs, ports and apertures for the placement of solution and instruments. The formation of these reservoirs, for example, for holding particular instruments therein, requires molding and extrusion processes which are expensive and complex. In addition, in use, the dental instrument must be placed in a specific hole or in a hole selected from a specific series of holes.

Another problem within the art is that sterilizing containers typically do not allow a practitioner to remove debris from the instruments placed within the containers. Instead, relatively large pieces of debris may remain disposed on the instrument while the instrument is placed in a sterilizing solution, hampering the sterilization process.

U.S. Pat. No. 5,967,778 in the name of Riitano attempts to solve some of the problems and disadvantages with the above mentioned prior art methods and devices. Notwithstanding, there remains a need for a hand held or small portable unit for sterilizing dental files wherein a container containing a disinfecting solution is sealed upon first use, disposable, and inexpensive to manufacture.

Another concern with known disinfecting devices and containers is the passing off of instruments and such as endodontic files from the dentist to the assistant. In passing off a file having a sharp end, for example, there is the risk that the dentist or assistant may puncture a finger if passing off is done too quickly.

By using the instant invention, this concern is obviated, as the dentist who is holding the file, merely has to insert it for a short period into the receiving end of the device. The receiving end is a large target, so precision control is not required.

Based on the foregoing, there is a substantial need within the art for a container which is configured to maintain a dental instrument in at least an aseptic condition. There is also a substantial need for such a container which features efficient cleaning of a dental instrument within the container. Additionally, there is also a substantial need for a container which can be repeatedly used during a single procedure, then discarded; wherein a new container is inserted into the device when another procedure is being performed on another patient. This quick and easy insertion of a new sealed sterile container into the actuator holder, allows for a quick change between patients in a busy dental practice, with almost no solution leaving the container or being able to spilled when discarded.

The more expensive part of the device, the actuator, is repeatedly used on one patient after another, without a requirement for sterilization, while the a new sterile container having an aseptic aqueous solution is inserted into the actuating part of the device for first use.

The synergistic combination of these two elements provides functionality not found in other commercially available devices.

Ultrasonic cleaning is well known in the dental industry as a effective way of cleaning tools and appliances to be used in dental procedures. Ultrasonic cleaning involves the use of high-frequency sound waves at about 18 kHz, or above the upper range of human hearing, to remove a variety of contaminants such as dirt, oil, grease, buffing/polishing compounds, and mold release agents, from form parts immersed in aqueous media. Some materials that can be cleaned are metals, glass, ceramics, and plastics. Ultrasonic agitation can be used with a variety of cleaning or disinfecting agents.

In the metals industry ultrasonic cleaning is often used for removing chips and cutting oils from cutting and machining operations, removing buffing and polishing compounds prior to plating operations, and cleaning greases and sludge from rebuilt components for automotive and aircraft applications.

Advantageously, ultrasonic cleaning is powerful enough to remove tough contaminants, yet gentle enough not to damage delicate tools or instruments. Furthermore, it provides excellent penetration and cleaning in the smallest crevices and between tightly spaced parts in a cleaning tank.

The use of ultrasonics enables the cleaning of intricately shaped parts with an effectiveness that corresponds to that achieved by vapor degreasing. Cavitation is a process by which partial vacuums in a liquid are formed by the application of high intensity waves or by a propeller disposed in that liquid. In cavitation, micron-size bubbles form and grow due to alternating positive and negative pressure waves in a solution. The bubbles subjected to these alternating pressure waves continue to grow until they reach resonant size. Just prior to the bubble implosion there is a tremendous amount of energy stored inside the bubble itself. The temperature inside a cavitating bubble can be extremely high, with pressures up to 500 atm. When implosion occurs near a hard surface, it changes the bubble into a jet about one-tenth the bubble size, which travels at speeds up to 400 km/hr toward the hard surface. The combination of velocity, pressure, and temperature the jet frees contaminants from their bonds with the substrate. Because of the inherently small size of the jet and the relatively large energy, ultrasonic cleaning has the ability to reach into small crevices and remove entrapped matter very effectively. It is the powerful scrubbing action and the extremely small size of the jet action that enable this to happen.

In order to produce the positive and negative pressure waves in the aqueous medium, a mechanical vibrating device is required. Ultrasonic manufacturers make use of a diaphragm attached to high-frequency transducers. The transducers, which vibrate at their resonant frequency due to a high-frequency electronic generator source, induce amplified vibration of the diaphragm. This amplified vibration is the source of positive and negative pressure waves that propagate through the solution in the tank. The operation is similar to the operation of a loudspeaker except that it occurs at higher frequencies. When transmitted through water, these pressure waves create the cavitation processes.

The resonant frequency of the transducer determines the size and magnitude of the resonant bubbles. Typically, ultrasonic transducers used in the cleaning industry range in frequency from 20 to 80 kHz. The lower frequencies create larger bubbles with more energy. The lower-frequency transducers will tend to form larger dents, whereas higher-frequency transducers form much smaller dents.

An ultrasonic cleaning system generally include a bank of ultrasonic transducers mounted to a radiating diaphragm, an electrical generator, and a tank filled with aqueous solution. A key component is the transducer that generates the high-frequency mechanical energy. An ultrasonic generator converts a standard electrical frequency of 60 Hz into the high frequencies required in ultrasonic transmission, generally in the range of 20 to 80 kHz. Many of the better generators today use advanced technologies such as sweep.

Ultrasonic tanks are generally rectangular and can be manufactured in just about any size. Transducers are usually placed in the bottom or on the sides, or sometimes both when watt density (watts per gallon) is a concern. The transducers can be welded directly into the tank, or watertight immerseable units can be placed directly into the aqueous solution.

U.S. Pat. No. 5,407,354 in the name of Fife discloses a multiple stage anti-microbial apparatus and method which uses a heated solution exposed to ultrasonic energy to treat dental instruments. Although Fife's device and method appear to achieve their goal, there remains a need for a device and system that will allow a dentist performing a root canal procedure to clean a file or similar instrument in a semi-automated fashion during the procedure and which does not require passing off the file to an assistant for cleaning.

The invention in accordance with this invention provides a device that will allow debridement of and between the flutes of a dental file used in an endodontic procedure, and will allow repeated use of that file in a same procedure within periodic debridement occurring as required. The prior art disclosed heretofore attempts to clean or provide an aseptic solution about the file end, but does not adequately remove debris lodged between the flutes of the file. When a file is only disinfected, but carries debris such as bone material, it will not adequately provide its function within the canal it is inserted into. On occasion, undebrided files bind within the root canal and break. Thus it is important to not only disinfect the file, but also to remove all or most of the debris bound to the file.

It is an object of this invention to provide both an aseptic solution as well as cavitation of said solution to remove debris from the file; furthermore, providing a device that can be used in situ is of paramount importance, as the device is required during an endodontic procedure.

Yet still further, this invention provides substantial disinfecting and removal of debris which can infect a technician or dentist in the unforeseen event of skin puncture with a file, during a procedure.

In accordance with this invention, there is provided an apparatus for cleaning dental instruments such as endodontic files or the like which need to be repeatedly withdrawn and replaced while performing a procedure such as a root canal on a patient, and wherein the dental instruments have a handle portion and a distal working end portion that needs to be cleaned and maintained in all aseptic condition both during use while repeatedly withdrawing and replacing the instruments during the procedure, the apparatus comprising:

a container having contained therein a disinfecting solution,

the container having about an upper portion thereof a cap for preventing the disinfecting solution from leaking out of the container; and,

a holder having a base with an opening for accommodating the container and an agitator coupled therewith for agitating disinfecting solution within a container when an instrument is inserted and withdrawn from the container so as to clean the dental instrument.

### SUMMARY OF THE INVENTION

In accordance with this invention, there is provided, an apparatus for cleaning dental instruments such as endodontic files or the like which need to be repeatedly withdrawn and replaced while performing a procedure such as a root canal on a patient, and wherein the dental instruments have a handle portion and a distal working end portion that needs to be cleaned and maintained in all aseptic condition both during use while repeatedly withdrawing and replacing the instruments during the procedure, the apparatus comprising: a container having contained therein a disinfecting solution,

the container having about an upper portion thereof a cap for preventing the disinfecting solution from leaking out of the container; and,

an agitator for agitating disinfecting solution within the container when an instrument is inserted and withdrawn from the container so as to clean the dental instrument.

In accordance with another aspect of the invention, there is provided, a kit for cleaning an endodontic file, for use with a vibrating agitator, said kit comprising a container containing a disinfecting fluid and containing loose abrading particulate matter, the container having at an upper end a stopper comprising one of a membrane, cap and a plug, for preventing the fluid from leaking out, and for wiping a working end of a dental instrument inserted therethrough and disposed within the container, the stopper allowing the dental instrument to penetrate repeatedly, without bending or damaging the working end. Preferably the particulate matter is an inorganic granular substance which will impinge upon the endodontic file when the vibrating agitator agitates the contents of the container.

In accordance with yet another aspect of the invention there is provided, a method for cleaning an endodontic tool during an endodontic procedure, comprising the steps of:

removing the tool from within the patient's tooth;

disposing the tool into a container having contained therein a disinfecting solution that is being agitated by an electronic agitator;

withdrawing the tool from the solution and during withdrawal passing the tool though a wiper at an upper end of the container, so as to clean left over debris off the tool; and,

re-inserting the tool in the patient's tooth.

In accordance with yet another aspect of the invention, there is provided a portable cleaner, for cleaning endodontic tools, comprising:

a container having contained therein a disinfecting solution,

the container having about an upper portion thereof a cap for preventing the disinfecting solution from leaking out of the container; and,

a battery powered holder for holding and providing agitation to the disinfecting solution when it is placed within the holder.

In accordance with yet another aspect of the invention, there is provided, a method for cleaning an endodontic tool during an endodontic procedure, comprising the steps of:

removing the tool from within the patient's tooth;

disposing the tool into a container having contained therein a disinfecting solution that is being agitated by an electronic agitator;

withdrawing the tool from the solution and during withdrawal passing the tool though a wiper at an upper end of the container, so as to clear left over debris off the tool; and,

re-inserting the tool in the patient's tooth

### BRIEF DESCRIPTION OF THE DRAWINGS

Exemplary embodiments of the invention will now be described in conjunction with the drawings in which:

FIG. 1 is an top view of the dental tool cleaning device in accordance with an embodiment of the invention;

FIG. 2a is a simplified side view of the dental tool cleaning device shown in FIG. 1 absent a transducer and circuitry;

FIG. 2b is a side view of a disposable container having cleaning fluid contained therein for placement within a recess of the dental tool cleaning device;

FIG. 2c is a side view of an alternative embodiment of FIG. 2b, wherein particulate matter is within the fluid within the container;

FIG. 3 is a side view of the container shown if FIG. 2a illustrating the transducer, control circuitry and a battery with charger; and,

FIG. 4 is a cross section of a preferred embodiment of the invention, wherein a container having a recessed bottom portion provides enhanced coupling with a disk-like transducer element.

### DETAILED DESCRIPTION

Referring now to FIGS. 1, 2a, 2b, 2c, and 3 the dental tool cleaning device is shown having a base 1 for supporting the sealed container 5 containing a disinfecting solution 12; the container 5 is inserted into an opening 2 in the base conforming to the outer periphery of the container 20. The container preferably has a handle or rim (not shown) for easy removal from the base 1. The base I also supports the container 5 as it rests upon and contacts ultrasonic transducers 7 shown in FIG. 3 permanently affixed within the side walls of the opening. Ultrasonic transducers for cleaning are commercially available from a variety of companies, such as Kyungwon Ferrite Ind. Co., Ltd. of Korea. Within a lower part of the base 1 is an electronic circuit 8 coupled to each ultrasonic transducers 7 via two leads, each pair labelled A. The circuit 8 is conveniently powered by a rechargeable removable battery pack 9 and provides a control signal to the transducer 26. An on off toggle switch switches is for switching the device on or off. A battery charger (not shown) is adapted to be disposed adjacent the battery 9 for charging 9.

The container 5 is preferably made of a disposable sterilized plastic material and must be rugged enough to withstand insertion into the base 1 and to withstand contacting and receiving energy from the transducers 7 when in use. By ensuring adequate contact between the transducer and the sealed container, energy from the transducer is transferred to the container. The container itself serves as a diaphragm coupled with one or more high-frequency transducers which vibrate at their resonant frequency due to a high-frequency electronic generator 8 source within which induce amplified vibration of the container 5. This amplified vibration is the source of positive and negative pressure waves that propagate through the solution 12 in the container 5. When transmitted through the solution 12, these pressure waves create the cavitation processes.

The solution 12 within the sealed container can be any aseptic solution for cleaning dental tools that is not harmful to patients, such as caustic soda, bleach, sodium hypochloride, or other commercially available aseptic solutions.

Optionally, a resistive heater strip 6 can be provided on the wall of the container 5, itself, or alternatively within the opening 2 of the base of the cleaner. The electronic circuit 8 can provide suitable current to heat the cleaning fluid within the container to enhance cleaning during the ultrasonic cavitation process.

The container is shown to have a resilient plug sealing the fluid therein at an upper end. The plug serves as a retainer for the instrument and as a wiper when the instrument is withdrawn from the container. Alternatively, instead of a plug being provided, a stretched rubber sheet seal similar to those used on medications to be withdrawn by a hypodermic needle can be used.

In operation, the tool cleaning device works as follows. A sealed container 5 containing a disinfecting liquid is placed within the opening 2 of the cleaner base 1, and the device is switched on by toggling switch 3 which energizes the electronic circuit providing a signal to the ultrasonic transducer. In the instance that the container having a heater strip is inserted, the ends of the strip make contact with a potential difference at two terminals contacting the ends from a voltage source (not shown), and the fluid begins to increase in temperature. A separate thermostatic switch can also be provided to control the heater circuit. After switch 3 is toggled to enable the transducer, ultrasonic waves are then generated through the solution in a usual manner. During a root canal dental procedure, the dentist removes a file with debris thereon, and inserts it directly through the lid 4 made of a rubber material, and the energized solution, through cavitation, cleans the file. As the file is then removed from the solution, the rubber lid also serves as a wiper, further ensuring no large debris remains on the file. Of course, other resilient inert materials can be used as lid materials, such as silicone.

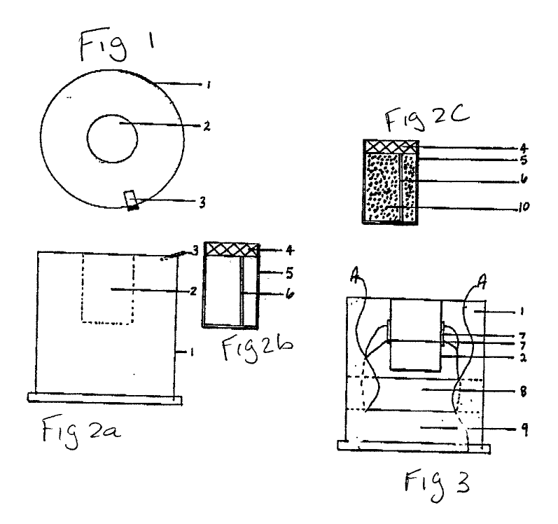

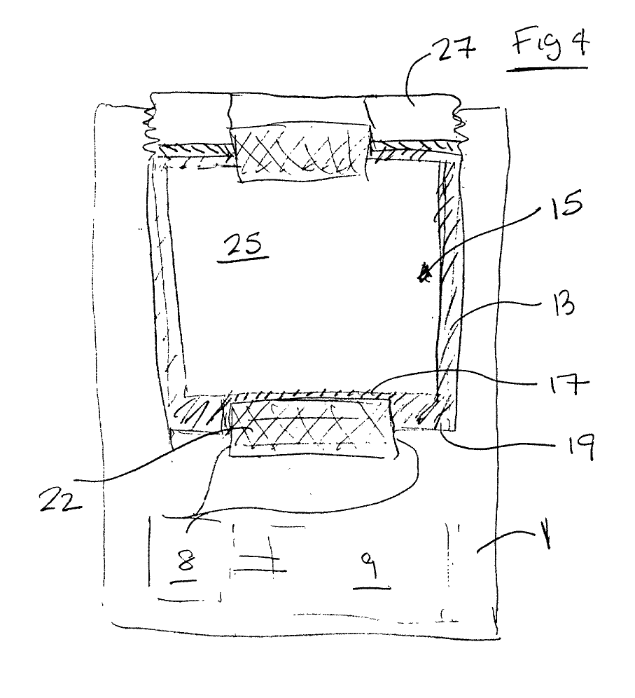

Preferably, ultrasound is employed to agitate the solution within the container 20; notwithstanding, other means can be used to provide agitation, such as an electro-mechanical vibrator, or other electro-mechanical vibrators or agitators. FIG. 2c illustrates an alternative embodiment of the invention, wherein particles of glass, sand or plastic beads are disposed within the aseptic fluid and used to impinge upon a file when mechanical agitation is provided, thereby removing debris. The plug sealing the container acts as a wiper ensuring that the particles and debris are left behind within the container.

A simple schematic of an ultrasonic cleaner can be found on the internet at http://www.repairfaq.org/REPAIR/R_samschem.html#SAMSCHEM_{—}041.

In a preferred embodiment of this invention, FIG. 4 illustrates a method of coupling an ultrasonic, or magenostrictive transducer with a container 15, such that enhanced coupling results. Typically, ultrasonic transducers, such as piezoelectric elements are attached to a fluid carrying container by way of being bolted, or via adhesives, or via solder. These means of coupling to the container has been unreliable over the long term, in many instances. Metalurgically attached transducers have been quite reliable, however require a metallic tank; In this instant invention, the container is removable from its base containing the transducer, a container 15 is shown if FIG. 4 having sidewalls 13 that are considerably thicker than the bottom wall 17, to thereby lessen unwanted vibration to the sidewalls. The bottom wall 17 is thick enough to withstand vibration from the transducer it is in contact with without cracking, but thin enough to transmit the vibration to the fluid. An annular periphery 19 of the bottom wall is thicker than the central portion contacting the piezoelectric transducer. Hence, when the container is secured down tight against the transducer 22, it effectively serves as a drum, or barrier from the cleaning solution 25, but is only a small impediment to the coupling of energy to the cleaning fluid from the transducer. A screw top O-ring 27 when tightly screwed-down ensures adequate contact between the transducer 22 and the thin centre portion of bottom wall 17.

Of course other embodiments may be envisaged without departing from the spirit and scope of this invention.

What is claimed is:

1. An apparatus for cleaning dental instruments such as endodontic files or the like which need to be repeatedly withdrawn and replaced while performing a procedure such as a root canal on a patient, and wherein the dental instruments have a handle portion and a distal working end portion that needs to be cleaned and maintained in all aseptic condition both during use while repeatedly withdrawing and replacing the instruments during the procedure, the apparatus comprising:
a sealed container having contained therein a disinfecting solution,
the container having about an upper portion thereof a cap for preventing the disinfecting solution from leaking out of the container; and,
an agitator for agitating disinfecting solution within the container when an instrument is inserted and withdrawn from the container so as to clean the dental instrument.

2. The apparatus as defined in claim 1, wherein the cap for preventing the disinfecting solution from leaking out of the container is a membrane about an upper portion of the container, and wherein the membrane allows the distal working end of the instruments to be repeatedly withdrawn and re-inserted into the container without bending, blunting or damaging the distal working end, and wherein the membrane substantially prevents fluid from leaking out if the container is tilted.

3. The apparatus as defined in claim 2, wherein the membrane is sufficiently strong and dense to clean debris from the distal working end of the instrument as it is removed from the container.

4. The apparatus as defined in claim 1, wherein the cap for preventing the disinfecting solution from leaking out of the container is a plug within an upper portion of the container, and wherein the plug allows the distal working end of the instruments to be repeatedly withdrawn and re-inserted into the container without bending, blunting or damaging the distal working end, and wherein the plug substantially grips the distal working end and prevents fluid from leaking out if the container is tilted.

5. The apparatus as defined in claim 2, wherein the membrane is sufficiently strong and dense to both clean debris from the distal working end as it is removed from the container.

6. The apparatus as defined in claim 1, further comprising loose granular material within the disinfecting solution which when agitation occurs by said agitator will impact against the distal working end of the instrument so as to loosen or remove unwanted debris and clean the working end.

7. The apparatus as defined in claim 2, further comprising loose granular material within the disinfecting solution which when agitation occurs by said agitator will impact against the distal working end of the instrument so as to loosen or remove unwanted debris and clean the working end.

8. The apparatus as defined in claim 6 wherein the loose granular material is an inorganic material.

9. The apparatus as defined in claim 8 wherein the loose granular material is one of: lass beads, plastic beads, sand, silica, plastic particles.

10. The apparatus as defined in claim 1, wherein the agitator comprises an electronically controllable ultrasonic transducer and control circuitry coupled therewith.

11. The apparatus as defined in claim 1 further including a heater for heating the fluid within the container.

12. A method for cleaning an endodontic tool during an endodontic procedure, comprising the steps of:
removing the tool from within the patient's tooth;
disposing the tool into a container having contained therein a disinfecting solution that is being agitated by an electronic agitator;
withdrawing the tool from the solution and during withdrawal passing the tool though a wiper at an upper end of the container, so as to clean left over debris off the tool; and,
re-inserting the tool in the patient's tooth.

13. A method as defined in claim 12, wherein the agitator is a vibrating agitator.

14. A method as defined in claim 12 wherein the container is a sealed container and wherein the tool is inserted through a sealing cover of the container.

15. A method as defined in claim 13, wherein the agitator is an ultrasonic agitator.

16. A method as defined in claim 13, wherein the disinfecting solution has therein, loose abrading particular matter, for impinging upon the tool as agitation occurs.

17. A portable cleaner, for cleaning endodontic tools, comprising:
a container having contained therein a disinfecting solution,
the container having about an upper portion thereof a cap for preventing the disinfecting solution from leaking out of the container; and,
a battery powered holder for holding and providing agitation to the disinfecting solution when ii is placed within the holder.

18. A portable cleaner as defined in claim 17, further comprising particulate matter including at least one of sand, glass beads, silica particles, sand, and plastic particles, disposed within the solution for impinging upon an endodontic tool disposed within the container when agitation is provided.

19. A device as defined in claim 1, wherein the container is disposable and wherein means are provided for securing the container within a base containing the agitator such that the container is fixedly held and secured against a transducer element.

20. A device as defined in claim 19, wherein the container has wall in one portion that are substantially thicker than in another portion, and wherein said another portion is contacting the transducer element when fixedly held.
